(19) United States
(12) Patent Application Publication
(10) Pub. No.: US 2025/0241562 A1
(43) Pub. Date: Jul. 31, 2025

(54) PORTABLE CATABOLIC MARKER MEASURING UNIT

(71) Applicants: Steven B. WALTMAN, Boulder, CO (US); Tylis Y. CHANG, New York, NY (US)

(72) Inventors: Steven B. WALTMAN, Boulder, CO (US); Tylis Y. CHANG, New York, NY (US)

(21) Appl. No.: 19/185,069

(22) Filed: Apr. 21, 2025

Related U.S. Application Data
(62) Division of application No. 17/047,684, filed on Oct. 14, 2020.
(60) Provisional application No. 62/658,765, filed on Apr. 17, 2018.

(51) Int. Cl. A61B 5/145; A61B 5/00; A61B 5/055; A61B 5/083; A61B 5/1455; G01N 33/483

(52) U.S. Cl. CPC A61B 5/14546; A61B 5/0022; A61B 5/055; A61B 5/083; A61B 5/14517; A61B 5/1455; A61B 5/4842; A61B 5/4875; A61B 5/6816; A61B 5/6824; A61B 5/6826; A61B 5/7239; A61B 5/7257; A61B 5/7275; A61B 5/7405; A61B 5/742; A61B 5/746; G01N 33/483; A61B 2576/02

(57) ABSTRACT

Health and care are improved with a portable catabolic marker measuring unit. In one example a unit includes a case, a catabolic marker sensor in the case to measure a concentration of a catabolic marker in a user in vivo, and a proximity sensor in the case to determine whether the catabolic marker sensor is near the user, wherein the catabolic marker sensor measures the concentration in response to the determining that the catabolic marker sensor is near the user.

### CROSS REFERENCE TO RELATED APPLICATIONS

The present application is a divisional application of U.S. patent application Ser. No. 17/047,684 filed Oct. 14, 2020, which is a national stage entry of International Application Serial No. PCT/US2019/027495, filed Apr. 15, 2019, titled Monitoring Catabolism Markers by Steven B. Waltman, which claims the priority benefit of U.S. Provisional Application Ser. No. 62/658,756, filed Apr. 17, 2018, titled Health and Better Care through Monitoring Health Markers by Steven B. Waltman, the priorities of which are hereby claimed.

### BACKGROUND

Modern medicine has taken two primary approaches to patient health. The first begins when a patient becomes aware of symptoms. The patient reports the symptoms to a doctor or other clinician who may then analyze the patient including looking for signs or inquiring about additional symptoms to determine a diagnosis. The diagnosis then leads to a specific treatment. This approach works well for conditions that have early and obvious symptoms. For conditions with mild symptoms or symptoms that only appear late in the onset of the condition, the patient may report the symptoms too late for effective treatment.

A second approach is to monitor some condition or sign within the body and then to apply a treatment to modify the monitored condition. The condition is usually not a disease or injury but, for the purposes of the treatment, is assumed to be related to the disease or injury. One common example is to monitor the presence of various cholesterols in the blood. Medications and dietary changes are prescribed to reduce the concentration of certain cholesterols. The concentrations are linked to heart failure and many patients have had their lives extended by cholesterol-reducing drugs. However, some patients with low cholesterol die of heart failure and some patients with high cholesterol do not have heart failure. While controlling cholesterol levels or some other physical condition improves the health of many patients, avoiding an emergency, controlling cholesterol levels does not address the detection of illnesses or the availability of care in an emergency.

Both of these approaches still require early detection and ready access to care. Today many people have easier and faster access to health care professionals and procedures. At the same time, no matter how easy and fast it is to reach a doctor or a clinic, it is not always obvious when a person should visit the doctor or the clinic. There are many cases of people arriving too late, after a condition or illness has become too grave. There are many cases of people checking into a hospital emergency room or urgent care center on weekends or evenings because the symptoms did not seem severe when a less expensive doctor or clinic was still open.

Over half of all hospital visits are unplanned so that the emergency room, which has the greatest cost, is a major source of hospital revenue. Many hospital admissions originate in the emergency room and then move on to some other part of the hospital. Hospitals are also allowed to charge more for an emergency room visit than for a planned admission. While hospitals make up a decreasing portion of the total healthcare cost, hospitals still provide all the most intensive care because of their unique capabilities and increasing consolidation. Over the last few decades, there has been increasing pressure to reduce medical costs, especially hospital costs. When hospitals cost more than clinics and when emergency room visits cost more than scheduled hospital admission, then resources can be saved by reducing emergency room visits.

This increased scrutiny on costs has been described as a shift from volume to value. One cost reduction effort has been alternative payment models that seek to reward health care providers for the value delivered to a patient rather than for the intensity of the care that is provided to the patient. In one aspect of this, hospitals are held accountable if a patient is readmitted to the hospital too soon after the patient was discharged.

Hospitals are reducing readmissions in many ways. One measure is to ensure that patients leave the hospital with correct discharge instructions and prescriptions. Another measure is to assign care coordinators who can help patients navigate post-acute care options and follow ups for 30 days after discharge. A more expensive measure is to assign home care nurses to visit patients early before a readmission. By checking up on patients at home, a problem may be addressed to avoid the patient being readmitted. Another measure is to assign the patient to a nursing facility either directly or when trouble occurs at home. The nursing facility can intervene to correct the problem instead of the hospital.

### BRIEF DESCRIPTION OF THE DRAWING FIGURES

The present invention is illustrated by way of example, and not by way of limitation, in the figures of the accompanying drawings in which like reference numerals refer to similar elements.

FIG. 1 is a simplified block diagram of a system for determining a health condition using a biochemical monitoring system according to embodiments.

FIG. 2 is a process flow diagram of an example of the operation of the system of FIG. 1.

FIG. 3 is a messaging diagram for central server health analysis according to embodiments.

FIG. 4 is a messaging diagram for local terminal health analysis according to embodiments.

FIG. 5 is a diagram of a tabletop NMR measurement system according to embodiments.

FIG. 6 is a process flow diagram of an example of the operation of the system of FIG. 5.

FIG. 7 is a diagram of a wearable measurement system according to embodiments.

FIG. 8A is a diagram of a portable measurement system according to embodiments.

FIG. 8B is an enlarged view of sensor of the system of FIG. 8A to measure an earlobe.

FIG. 9 is a diagram of an alternative portable measurement system according to embodiments.

FIG. 10 is a diagram of a fixed urea measurement system according to embodiments.

FIG. 11 is a block diagram of a computer system suitable for embodiments.

FIG. 12 is a diagram of components of the system of FIG. 7 according to embodiments.

FIG. 13 is a diagram of a Raman spectroscopy system according to embodiments.

FIG. 14 is a diagram of an alternative Raman spectroscopy system according to embodiments.

### DESCRIPTION OF EMBODIMENTS

As described herein, biochemical signatures are used as an early warning indicator to identify the onset of a wide range of illnesses before they become acute. The early warning can be used to schedule a visit and avoid the emergency room. The early warnings may also be used to schedule treatment before the condition progresses too far and it is too late. This affects a market that makes up 2% of the United States Gross Domestic Product (GDP). The biochemical signatures may be tracked and monitoring using conventional medical equipment or with specialized devices. A small home device may be used for constant or frequent non-invasive monitoring. The device may also be configured with communications to connect people to health professionals if there is a sign of trouble.

Hospital and urgent care clinic healthcare is ripe for disruption. One of the most painful entry points into the medical ecosystem, emergency care, is also addressed herein. As described herein, signs of the health of a patient are monitored frequently and, in some cases, non-invasively to determine when the patient's health is deteriorating. Such a determination empowers patients and providers to be more than reactive players in the struggle against many forms of illness. Early detection and communication allows the experts, such as doctors, clinicians, and other healthcare providers, to initiate contact and direct care with the appropriate tools and with immediate feedback. Rather than focus on any single disease or a symptom of a disease, many of the signatures monitored as described herein indicate many or all types of illness across the body's systems.

The described methods and systems apply to all who are interested in risk stratification and early detection. One application is to reduce hospital readmissions. This is an area of urgent need and of recent alignment between good clinical practice, policy and financial pressures. By keeping patients out of the hospital when they don't need to be there, hospitals can increase ratings and payments, improve patient outcomes, and care for more patients in need. This is increasingly true for an aging and longer-living population.

Other applications are for care management companies, insurers, and employers. These groups are all interested in keeping patients and employees healthy and in keeping costs down. Similarly, nursing homes and other long-term care facilities want to keep their patients out of the hospital. Everyone is interested in determining when patients need treatment before it is too late. By detecting serious conditions before the symptoms become apparent, emergency room admissions are avoided and regularly scheduled care can be used instead.

In another emerging trend, the market is flooded with fitness monitors or trackers and smart watches. These devices typically measure a heartbeat rate and overall motion of the wrist. Some are able to measure other electrophysiological phenomena on a wrist or torso. Such devices can be helpful for fitness training and to monitor activity during sleep, but they measure an exercise level. They do not indicate illness. The fitness monitor does not provide any baseline for health versus fitness but a baseline of resting body activity versus active body activity.

A different monitoring trend is to use very targeted testing aimed at specific conditions. The measurements are repeated and the aim is to determine whether the patient has recovered from that specific condition. This type of monitoring requires specific high-intensity, frequent, human-error riddled interventions. This type of care management is a very manual and high intensity proposition. Care coordinators and nurses check up on individual patients in a non-scalable and expensive way. The tools for diagnosis and alerting are rudimentary.

In contrast, the methods and systems described herein allow patients or nearby health care providers to repeatedly and reliably monitor one or a few conditions to measure overall health. In some cases, a different baseline can be established independently for each patient. Variations from this baseline can be used to trigger an alert or warning of some kind.

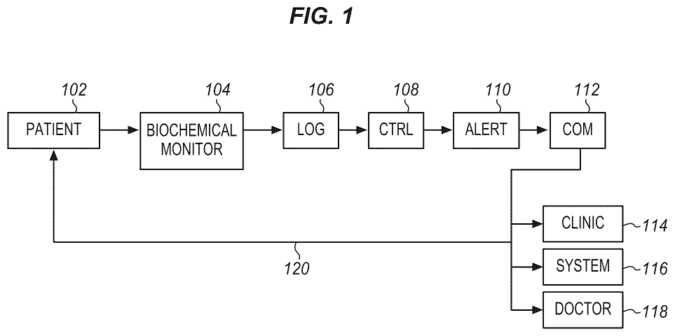

FIG. 1 is a simplified block diagram of a system for assessing generalized health using a biochemical monitoring system. In this example, a patient 102 accesses a biochemical monitoring system 104 to allow a value for one or more particular biochemical markers to be measured. The monitor may be a fixed or portable device or a wearable device. The device may require the patient to perform particular operations or the device may operate autonomously or automatically. As an example, the device may require the patient to insert a finger into a scanning device and hold the finger there for some time duration. As another example, the device may be worn on a wrist or in another location or be attached to or part of a garment and perform measurements at appropriate intervals. As another example, the device may be operated by a technician or health care provider.

The monitor 104 generates values for one or more biochemical markers and provides these to a log 106. The log stores multiple measurements over time. The log is made available to a processor or controller 108 that analyzes the new log entry in light of previous log entries and then generates an alert 110 that is communicated by an alert transmitter or communications interface 112. The transmitted alert may be used only to identify an abnormality, or there may also be alerts for normal results. In some cases, the alert is used only to indicate that the patient should be checked, while in other cases, an alert is also issued for good or stable health. The log allows measurements to be compared over time. For most markers there will be a healthy level and an unhealthy level. These levels may vary for different patients. The log allows the unhealthy level to be identified as a variation from a normal healthy range of levels. When the marker is used to evaluate recovery, then the marker may instead be monitored to determine whether health is improving as compared to the initial state.

In the example of FIG. 1, the alert condition is determined at a controller 108 that is attached to the log. This controller may be a part of a local device that is acting with the monitor or is a part of the monitor. Alternatively, the controller may be coupled to a server system 116 or another system through the communications interface 112 that provides additional information for use in determining an alert. As a further alternative, the information may be sent to the server system or another remote device to determine the alert. In this case, the controller receives the remote determination and then issues the alert accordingly.

The alert is generated and then sent to one or more different entities as appropriate depending on the particular implementation. The alert may be sent to the patient 102 on a direct line 120 or indirectly through other recipients. The alert may also be sent, for example, to a clinic or hospital 114, to a server system 116, to a doctor 118 in charge, or to any other appropriate person involved in the patient's health. Alerts may also be sent to friends and family.

The server or analysis system 116 stores and analyzes the received data. The system may be used to determine the seriousness of the alert and then to determine any other parties to alert such as the doctor and clinic. If the system receives data from many other patients, then the data may be analyzed for trends and to determine health baselines. A wide variety of data analytics may be applied to determine and analyze patterns as they occur. The alert may be used to establish a communication between the doctor 118 or clinic 114 and the patient 102. The communication may take the form of a request to schedule an appointment or a request to perform additional tests. In other words, if the alert indicates that the patient is ill or deteriorating, then the doctor or clinic may notify the patient to arrange for an examination. The appointment may be used to determine an early diagnosis and establish a treatment plan.

In some embodiments, the alert does not indicate any particular illness or disease. The next step in the process is to collect more information. The patient may gather some diagnostic information individually and provide that to the doctor or clinic. The patient may report to a local clinic or office where diagnostic information may be gathered. The patient may also or instead meet or communicate with a doctor or other professional to perform additional measurements and obtain a diagnosis. Unlike many systems, the presence of an alert condition does not indicate the presence of a particular disease. No particular disease is being monitored. Instead, the alert indicates a general amount of health or illness and the next step is to diagnose the patient to determine the cause of the alert.

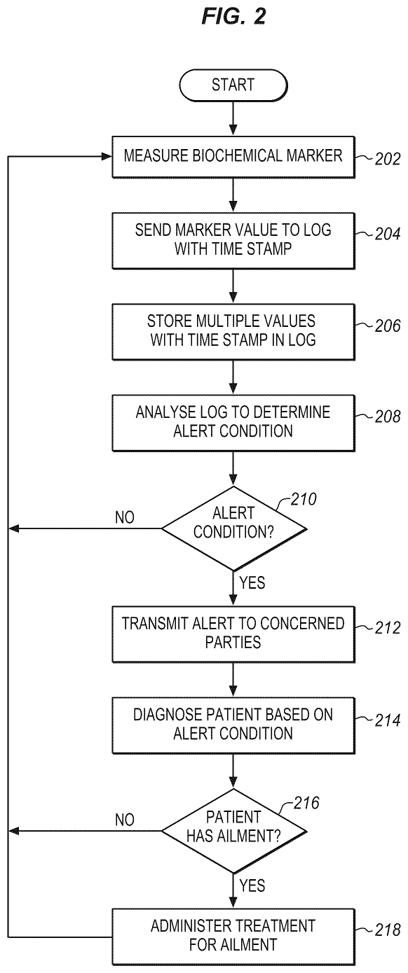

FIG. 2 is a process flow diagram of a particular example of the operation of a system such as that of FIG. 1. FIG. 1 starts with a measurement of a biochemical marker in or on the patient's body. In many examples, the marker may be the urea concentration in a patient. However many other markers may be used as described in more detail below. Urea is a soluble crystalline nitrogenous compound that is generated when proteins are decomposed in a human body. It is found chiefly in urine but also in blood, saliva, and other bodily fluids. Since urea is generated by decomposing proteins, the concentration of urea in the body increases as the body's catabolismrate increases. Catabolism is the destructive part of metabolism that involves the generation of energy from proteins to support vital processes and activities. Urea may therefore be used to determine the energy demands that are being supplied by the body. This can be compared to a patient's physical activity level.

Each person will have a normal, typical, or usual range for urea concentration in any particular part of the body. This normal range reflects a healthy metabolism and normal bodily functions. The concentration will vary during times of high or low activity and around meals. Urea concentration is one example of a catabolic marker that is easy to measure and that also indicates abnormal health. When the urea concentration is high but the patient is not exercising or finishing a meal, then the body's metabolic activity is high for another reason. A common reason is that the immune system or some other protective or regenerative body system is more active than normal. A high urea concentration is used herein as an indication that there is a foreign disease agent at work or an internal injury stressing the body. The catabolic marker may not be able to distinguish between a fever and a bruised spleen, but it will detect both events as an unexplained increase in body activity.

At 204, the measured urea concentration or other marker value is sent to a log 106 or other storage device with a time stamp. The measurement and logging are repeated so that the log has a history of measurements that have been accumulated over time. At 206, the log stores the measurements with the respective time stamps. This allows the measurement history to be made available for analysis. The log values are analyzed at 208 to determine whether there is an alert condition. Different alert conditions may be supported. There may be an alert if the patient is normal or healthy or consistent. There may be an alert for a variation from normal and there may be alerts for differing amounts of variation from normal. The analysis may be at a local controller or processor 108 or remote, or using combined local and remote resources. Processes for analyzing the data are described in further detail below.

A variety of different alert conditions may be used alone or in various combinations. At the simplest level, the alert indicates whether the patient is healthy or sick and if sick, it may also indicate how sick. This amount of sickness may be referred to as an illness value. The level of sickness or illness value may be used to determine how quickly medical care will be provided. For such an alert to work, the system might determine what is normal and it might also isolate or compensate for other factors that influence the catabolic rate but that are not sickness. In one example, the patient is first measured multiple times when the patient is known to be healthy. These measurements may be used to establish a healthy range for that particular patient. Any variation outside of that range indicates that the patient may not be healthy. After an alert and a diagnosis, if the patient was diagnosed as actually being healthy, then the healthy range that is used to determine the illness value or illness alert may be adjusted. To isolate other factors, the patient may provide the measurement at the same times each day and select times that are not near exercise and meal times. Alternatively different healthy ranges may be determined for different times of day. The alert for such a case may be generated simply when the urea concentration is outside the normal range.

A baseline or regular pattern may be determined using the multiple stored measurements. The log entries for each measurement allow the measured marker values to be compared over time. A baseline level or regular pattern may be established using the log entries. The baseline or patter may then be used to correct for diurnal or cyclical fluctuations in the marker for example including diet, exercise, and medications. The difference between a more recent measurement and the baseline, pattern or previous measurements can be compared to a threshold. A log entry that is different by more than a threshold corresponds to an alert condition. A more complex approach using the pattern is to compare the log entries to find a regular pattern. An alert or an illness condition is determined when a log entry does not fit the regular pattern.

As a further alternative, the alert condition may be determined by analyzing the first, second, or higher derivative of the measurement over time to determine differences in the recent measurement over the baseline level or the regular pattern. Another approach to eliminating diurnal or other cyclical fluctuations is to apply a Fourier transform to the stored measurements and then remove the cyclical variations. As another alternative the stored log entries may be rendered as an image. Image recognition techniques may then be used for the image to detect distinctive illness patterns in the image.

As explained above, different approaches may be used to determine an illness condition or illness value using the values stored in the log over time. The biochemical sensor and signal processing applied to the sensor signals may be used to provide cleaned, normalized spectra, highlighting the marker's signal such as the urea concentration. To detect illness, at a simple level, the relative change of the marker signal or concentration can be monitored over time. This can be done by assessing the time rate of change such as a percent increase or decrease per hour. The first order time rate of change eliminates many simple noise sources in the signal. To improve sensitivity and specificity, the second order (and even higher order) derivatives over time may be assessed. This may yield improved discrimination over normal variations.

The accuracy may be further improved by also removing systematic cyclical variations through, e.g. a Fourier transform. Cyclical variations may also be removed by assessing a patient's diurnal patters and correcting for these.

Image classification techniques such as those prevalent in artificial intelligence systems (e.g. Resnets, convnets, GANs, etc.) may be used by rendering the log values as an image. The image is made more detailed by including other signals, such as those created through measured lactate concentration values. One type of image defines the X-axis by wavenumber and the Y-axis by time. The image classification system may be tuned to discriminate a true signal and remove spurious biological as well as system collection noise.

At 210 if an alert condition or illness condition is determined, for example if the urea concentration is outside of the normal range, then an alert is generated for that condition. At 212 the alert is transmitted to concerned parties, such as the patient, a friend, a caretaker, the doctor, a clinic, a hospital, or one or more of these and other parties. If there is no alert condition, then the process returns to 202 to wait for more measurements from the monitor. At 214 the patient with the alert condition is diagnosed to determine an ailment. If an ailment is found at 216, then at 218 a treatment is administered for the determined ailment. The process may then be repeated from START. If there is no ailment, then similarly, the process may be repeated from START. If the system generates frequent false alerts then an evaluation may be necessary to determine whether there is a fault in the measurement at 202, in how the measurements are evaluated at 208 or in some other part of the system.

The described system and method may in many cases be more sensitive than a patient in detecting a malady that should be diagnosed. The system therefore may cause the patient to submit to a diagnosis or schedule an appointment earlier than the patient otherwise would. As one example, if a patient has an infection, the patient may not be immediately aware of the infection. At the same time, the immune system will be activated to fight the infection and the catabolic level will be increased. This can be detected by the biochemical marker measurement tool and alerted to the patient or doctor. As a result, the infection can be treated several days earlier during normal office hours instead of being treated after the level of infection has reached a critical state and the patient is concerned that something more serious is wrong.

An infection is one common example, but the same or a different catabolic marker may indicate many other maladies before the patient is aware of the condition. In some cases, there may be no symptoms that the patient can perceive and yet the catabolic marker will indicate an unhealthy condition. Some ailments do not have strong symptoms or any symptoms and other ailments have symptoms that are similar to other common conditions. There are also some patients that are not particularly sensitive to disease symptoms and may not recognize the symptoms even when they are present. The catabolic marker will overcome each of these situations.

For the hospital readmission situation described above, the patient may be monitored as in FIG. 2 so that both the patient and the hospital may be alerted if the patient's condition degrades or does not improve. Typically, upon leaving a hospital, the patient's catabolic rate will be high. However, as the patient's condition improves after being discharged, the catabolic rate should decline. If the concentration of the catabolic marker does not decline or if it increases, then a person can be sent to the patient to investigate the patient's condition. The treatment may be adjusted. The patient may be sent to some other clinic to treat the condition, or the patient may be readmitted to the hospital. In some cases, the expected catabolic rate may be adjusted to prevent false alarms. This may result in fewer readmissions and when there are readmissions, the readmission will be sooner so that the patient's condition is better and may be treated more effectively and for less cost.

While the concentration of urea is used in the example above, there are many other biochemical substances that naturally occur in the body and that may be used as markers. Urea concentration is a product of and therefore an indicator of the nitrogen cycle of catabolism as mentioned above. There are other products of this cycle such as uric acid, lactic acid, and ammonia. There are also proteins and enzymes that are released with catabolism, such as LDH, CK, AST, ALT, and others.

Instead of or in addition to catabolism, markers for other natural body processes or cycles may be measured. The body generates several different compounds that may be used as inflammation markers, such as WBC, acute phase reactants such as CRP, complement, fibrinogen, a2-macroglobulin, ferritin, etc. These markers indicate that the body is suffering from an inflammation but do not give any indication as to where and how the inflammation is occurring. Instead of inflammation, hydration may be measured using hydration status markers, such as total protein, albumin, osms, etc. Instead of, or in addition to catabolism markers, alanine cycle markers may be used such as alanine, a-ketoglutarate, b-hydroxy butyrate, etc. The alanine cycle is a hydrolysis of proteins in the body that occurs during transamination reactions.

Other potential markers include amino acids such as glycine and valine. Increased glycine levels are associated with inadequate nutrition, which may be caused by illness. Valine is associated with insulin resistance and diabetes. These and other markers may also indicate muscle or tissue breakdown from exercise or other causes.

These various biochemical indicators are found in many different places in the body and can be detected and measured in many different ways depending on where they are detected. Urea, uric acid, ammonia, and many other catabolism markers are found throughout the body. The measuring device may be directed at saliva, sweat, urine, breath, blood or other body fluids using a transdermal scan or probe, or a scan or probe directed to the sclera, retina, or other eye areas, or any other suitable location.

The measurements may be taken by analyzing fluids that have been collected and placed in a special chamber. This chamber may be like a spittoon or a specially adapted toilet that includes sensors to analyze for urea and other compounds. In some cases, the fluids may be analyzed without a special chamber. A device may be placed and worn that is in contact with the wrist, forehead, or some other body area using smart clothes, smart shoes or wearables. The device may use tiny probes to support a subdermal sensor or for transdermal measurements such as electrophoretic measurements, differential measurements of electrical resistance using alternating current or pulses, or other measurements. In some cases, a sensor may be implanted in the patient subdermal or subdural to measure biochemical markers. In some cases, a smart pill may be used that is taken internally and passed through the body. The smart pill may be configured to measure catabolism markers or other biochemical markers such as those mentioned above.

Instead of or in addition to collecting fluids, the patient may be measured using a free-standing or separate measuring device. As mentioned above, a device may be configured to receive a patient's finger into a chamber. The device may then perform transdermal measurements on the finger, such as a transdermal spectroscopy with an optical system. At the same time, such a device may also collect sweat from the finger, measure pulse rate, oxygen levels and other physiological data. A different type of optical eye scanner may be used for the eye to measure scleral reflectance, blood vessel analysis, etc. An eye scanner may also be able to determine pulse, blood pressure and other physiological parameters of the patient. These devices may be free-standing independent devices or they may be coupled to and operable through a patient's computer, smart phone, medical terminal, or another type of device.

There are a variety of different ways to measure the presence or concentration of biochemical markers in a patient's body. In some embodiments, Raman spectroscopy is used. This may be used to measure urea concentration in vivo. It may be used to measure markers through the skin by contact with the skin (contact transdermal spectroscopy) and it may be applied to collected fluids in a container such as the spittoon or special configured toilet mentioned above.

Raman spectroscopy relies on the Raman Effect by which light is absorbed by the sample and then re-emitted at a different frequency which is shifted up or down from the absorbed frequency. Raman spectroscopy uses a monochromatic absorption light, typically in the near infrared range or visible range, so that all of the re-emitted light is shifted up or down from the single frequency of the absorption light. The re-emitted light is captured and the frequencies and amplitudes of the re-emitted light are analyzed to determine the presence of various compounds in the sample. The amplitudes of the frequencies indicate concentration levels. Raman spectroscopy can be performed with a monochromatic probe laser to illuminate the sample, an image sensor to record the re-emitted frequencies and their relevant amplitudes and a processor to analyze the recorded frequencies. Optical and container systems are used to direct the absorption light and to collect the emitted light. In some of the examples herein, a patient's finger, ear lobe, etc. is used as the container for the sample and the optical system directs the laser through the skin and collects re-emitted light through the skin.

The magnitude of the Raman signal is increased near an appropriate surface. This is called Surface Enhanced Raman. A subdermal implant of an appropriate material may be used to further increase the Raman signal.

The signal from the detector in Raman spectroscopy or other measurements may contain interference in addition to the desired signal. This interference can arise from ambient light, noise in the detector, or other sources. The Raman probe laser can be modulated so that the signal arising from it can be differentiated from the interference.

While Raman spectroscopy may be well-suited to transdermal applications using commonly available components, in part, other measuring techniques may also be used. Similar hardware may be used for far-infrared and mid-infrared spectroscopy. Plasmon resonance is another optical technique for sensing compounds. Fluorescence based nanotube technology may also be used to detect compounds. In other examples, a selective ion probe may be used to detect some small molecules, such as an embedded urease. Nuclear magnetic resonance (NMR) is a sensing technology that may be used to detect complex or heavy targets such as protons, 14N compounds and 15N compounds. Quadrupole NMR may be used for the detection of compounds containing 14N. NMR may be used to sense compounds in vivo. Magnets may be used for measuring electrophoretic effects and mass spectrometry may be used to detect volatile organic compounds in the breath or other fluids. For a larger measuring device, a bacteria cytometer or any type of wet chemistry may be used to analyze the concentration of various biochemical markers.

As shown in FIGS. 1 and 2, the inventive operations may be viewed as having three basic aspects. First, there is the taking of measurements using the monitor. A variety of different monitor devices and different possible biochemical markers are described herein. Most of the described biochemical markers vary in concentration with the amount of catabolism in the patient's body. Accordingly, in many embodiments, the first aspect is to determine the patient's catabolismrate.

The second aspect is to determine a level of illness or health based on the measured catabolismrate (or based on another biochemical measure). This determination may be done locally by the monitor device or a connected computer. Alternatively, the determination may be done at a central server and processing system. This allows for data to be collected from multiple patients so that various artificial intelligence, data analytics, trend analysis, and other techniques may be applied. A third option is for the analysis to be done locally and centrally.

The third aspect is the action that is taken based on the results of the analysis. In a simple analysis, the result will be a further examination. A catabolism marker is an indication of overall health, not an indication of a particular ailment. High, or excessively low, catabolism does not determine what must be done for the patient so the next step is to examine the patient more fully to determine what type of treatment, if any, is suitable. As described above, an attending physician, or responsible clinic can notify the patient to come in for an examination. If the monitoring is being done in a post-surgical context, the patient might be readmitted to the hospital or to an outpatient facility. If the monitoring is being done in a clinic, then it may be a matter of notifying appropriate staff of the clinic to come and check on the patient.

The described system and method allow for much higher precision and better analysis than before. This is due, in part, to the frequency of the measurements, the time and date stamps associated with the measurements, and the ability to receive measurements from many different patients at a central server. A further enhancement is to provide results from the scheduled examinations to the central server system. With this kind of information, trends and patterns may be identified so that a patient may be deemed healthy based on the patient's typical catabolism variations through the day. Another patient may be deemed unhealthy if the catabolism pattern is within a healthy range but otherwise matches that of patients that were diagnosed as unhealthy during an examination.

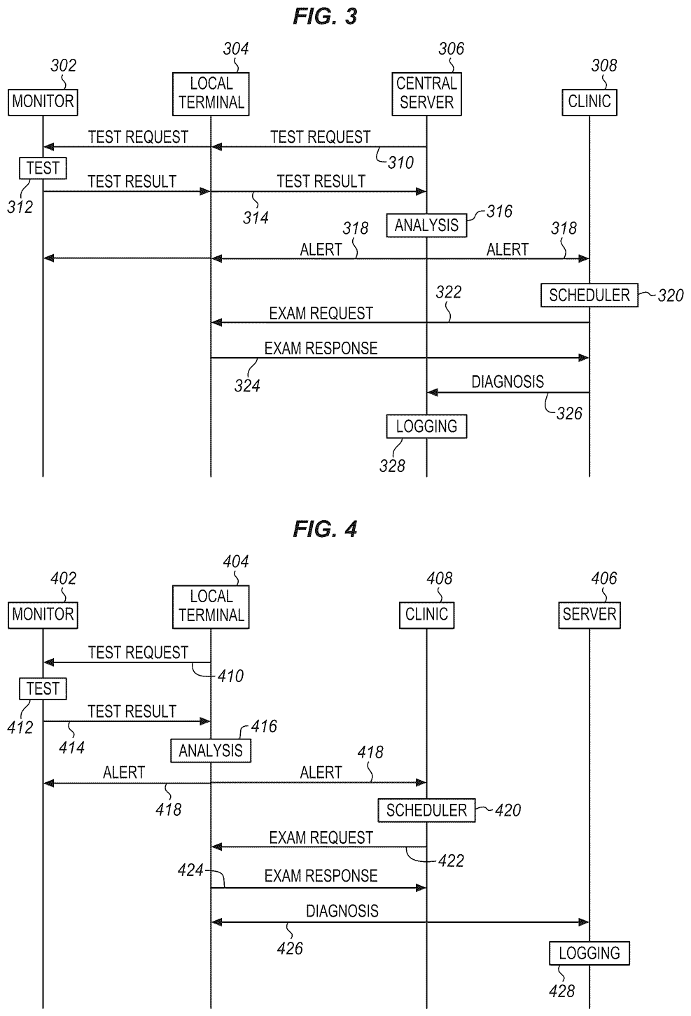

FIG. 3 is a messaging diagram to show a sequence of messages and actions in accordance with embodiments of the present invention. The terminals or messaging nodes in this configuration are similar to those shown in FIG. 1. In this example, there is a monitor 302 that performs tests on the patient to measure an amount of a biochemical marker, such as a catabolism marker. The monitor is connected to a local terminal 304 to receive the test results and forward the results to a connected central server 306 that accumulates all of the test results for this patient and many possible other patients. A clinic 308 is coupled to the central server to receive test results and examine the patient.

A process begins with a new test being performed on the patient by the monitor to determine an amount of a biochemical marker. At 310 a test request message is generated. In this example, the test request is generated by the server system and sent to the monitor through the local terminal. The test request may instead be generated by the monitor or the local terminal. The test request may be based on a schedule, such as time of day or based on other information from the central server or the clinic. In response to the test request a test is performed at the monitor at 312 to measure an amount of the marker. The test result 314 is sent to the central server through the local terminal.

The central server analyzes the result at 316 to determine whether an alert condition is presented by the test results. If so then an alert 318 is sent to the local terminal, to the monitor, to the clinic and any other relevant terminals in the system. The conditions for sending the alert and the recipient of the alert may be adapted to suit different implementations and circumstances. In this example, the local terminal is acting as a communications and messaging node for the patient so that test requests and appointments may be made through the local terminal. In other examples, a different messaging node is used for these purposes.

The clinic, upon receiving the alert, activates a scheduler at 320 to determine an appropriate time to examine the patient. The particular action taken by the clinic and the urgency of the action may be determined by the nature of the alert. Some alerts may be stored for later reference, while other alerts may require immediate attention. The scheduler, in this example, schedules an examination and sends an examination request 322 to the local terminal. The patient may consider and respond to the request. The local terminal sends the response 324 to the clinic and then the patient attends the appointment at the clinic where the examination is performed. In some cases, the alert may be more urgent or severe so that the patient is examined at a hospital. The clinic may be available to schedule the hospital examination or the examination request may indicate that the patient should schedule the examination with the hospital. A similar approach may be used to schedule other types of examination outside of the clinic. In other cases, such as post-surgery monitoring, the clinic may be a hospital

The examination results in a diagnosis which is sent at 326 to the central server. The central server logs the test result and corresponding diagnosis at 328. The log may be used in the analysis of this and other patients in response to other test results. The diagnosis may be that the patient is healthy or that the patient has a particular ailment. The diagnosis may also include an indication of the severity and urgency of the condition. All of this information may be compared to this and other test results at the server system to better determine how to analyze later test results and the types of alerts to send.

FIG. 4 is an alternative messaging diagram to show a sequence of messages and actions in accordance with different embodiments of the present invention. In this example, there is a monitor 402 that performs tests on the patient connected to a local terminal 404 to receive the test results and forward the results to a clinic 408. The clinic is coupled to a central server 406 that serves as a records repository. In this example, the analysis and test scheduling are performed by the local terminal which may or may not be integrated with the monitor. The clinic performs the same operations but connected to the local terminal instead of to the central server.

A process begins as a test request message 410 that is generated and sent to the monitor. In this example as in FIG. 3, the test request is optional. A test may be initiated in any of a variety of different ways including by the patient or a technician. A test is performed at the monitor at 412 in response to the test request to measure an amount of the marker. The test result 414 is sent to the local terminal where it is analyzed at 416.

The local terminal may use data received from external sources, data that it has compiled over time, or any other data as explained in more detail to analyze the test result at 416 to determine whether an alert condition is presented by the test results. If so then an alert 418 is sent to the clinic. The alert may also be sent to the monitor in some cases to alert the patient. In this example, the local terminal is acting as an interface to the patient as well as a control terminal for the process. Alerts and appointments are accordingly arranged through the local terminal.

The clinic 408, upon receiving the alert, activates a scheduler at 420 to determine an appropriate time to examine the patient. The appointment time may depend upon the nature of the alert. The scheduler at the clinic schedules an examination and sends an examination request 422 to the local terminal. The patient may consider and respond to the request. The local terminal sends the response 424 to the clinic and then the patient attends the appointment at the clinic where the examination is performed.

The examination results in a diagnosis which is sent at 426 to the local terminal. The results may also be sent to a central server 406 that logs the test results and corresponding diagnosis at 428. The log may be used for record keeping, data analytics, or a variety of other purposes as described herein. While in the example of FIGS. 3 and 4, the result is indicated as being an examination and diagnosis, other actions may be taken in response to the test result. One action is to log the data and then wait for the next test measurement. Another action is to request another test. The test result may be far enough outside of normal boundaries that it should be confirmed by being repeated. Alternatively, other tests may be performed to verify a suspected condition of the patient. Instead of an examination request, the clinic may send a survey to the patient through the local terminal to allow the patient to provide any symptoms or signs to the clinic. The survey may then be used to determine whether an appointment is necessary.

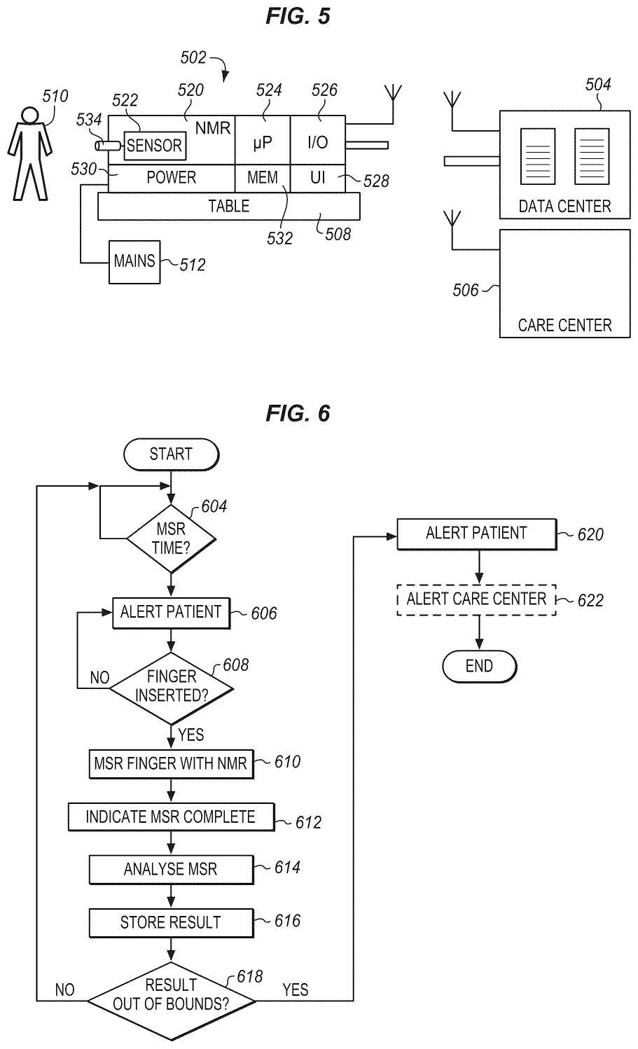

FIG. 5 is a diagram of a tabletop NMR urea measurement system coupled to the data center or server system and a care center, clinic, or hospital according to another embodiment. The tabletop unit 502 provides fast and easy patient 510 health monitoring. It may be complemented optionally by a data center 504 to analyze the results and a care center 506 to provide any care that may be appropriate based on the measurement. Alternatively, or in addition, the measurement unit may also act with the patient 510 directly and then alert the patient when action should be taken based on the measurement.

In this embodiment, the instrument is a tabletop unit. Here a table 508 supports an NMR instrument 520 that has a sensor 522 for NMR measurement. The sensor is controlled by a microprocessor or controller 524 that conducts the measurement and determines the result. The microprocessor stores the measurement in a memory 532 where it is ready to be transmitted through an input/output (I/O) interface 526 that may be wired or wireless. The instrument is powered by a power supply 530 coupled to the mains 512. This may be backed up by a battery system within the unit. A larger battery may be used to allow the unit to be transported to a patient and operated temporarily away from the table.

The instrument also has a user interface 528 that may be used to alert the patient to perform a measurement, to allow the patient to perform the measurement, and to provide results to the patient. The user interface may have an activation switch, and a status indicator, such as an LED, multiple colors of LED, or any other suitable display and buttons including a touch screen display and an audible alert. In one example, measurements are made at regular intervals. The measurement unit provides an alert through the user interface that it is time for a measurement. The processor may generate the alert based on an internal calendar or timer or by receiving a command externally through the I/O interface. The alert may be in the form of a beeper, a lamp, or a display on the user interface. The I/O interface may also send the alert to the patient using a Wi-Fi®, Bluetooth®, SMS, or other interface to a computer, tablet, phone, wearable, or other suitable device.

Upon receiving the alert, the patient comes to the instrument and performs the test. In this example, the NMR measurement includes a sensor tube 534 with an opening at one end to allow the patient to insert a finger into the tube or cylindrical sleeve of the measurement unit. Depending on the particular implementation, the patient may insert a finger, earlobe, toe or other suitable part of the body into the spectrograph and push an activation button of the UI or wait to be detected. The spectrograph may alternatively be configured to be placed beside the skin to measure a wrist, forehead, or some other body part. The finger may be automatically detected or the patient may provide an indication such as a button press to the user interface.

The measurement unit then conducts a suitable measurement of a suitable sign. In one embodiment, an electromagnetic pulse inside the cylinder perturbates the nuclear spins of atoms in the patient and the resulting emitted echo from the patient is detected by a pickup coil or a similar device. Magnets generate a constant magnetic field during this process. The detected signal is then analyzed to determine a presence of and amount of one or more biochemical markers. When the measurement is complete, the user interface may provide an audible or visible indication to the patient and the patient may remove the finger.

In this example nuclear magnetic resonance spectroscopy is used as the measurement method. Permanent magnets around the finger cylinder generate a magnetic field that is applied to the finger. The probe coil of the measurement unit around the finger cylinder applies electro-magnetic waves to the finger and measures the electro-magnetic response of the finger to provide the measurement result. A variety of isotopes may be detected in this way, including isotopes of Nitrogen, Oxygen, and Sodium. In this case, the Nitrogen isotope 15N is used to measure a chemical shift that is characteristic of urea. The magnetic response of the finger is analyzed by the internal microprocessor to determine the concentration of 15N. This is used to infer a concentration of urea which may be used as a general healthiness sign.

Standard NMR only detects nuclear isotopes with an odd atomic number. Natural nitrogen is 99.6% 14N, and 0.4% 15N. Urea has two nitrogen atoms, which renders it easier to detect than other compounds but in most cases, both atoms will be 14N and not detectable. The presence of two nitrogen atoms help because it gives two chances for one of them to be 15N. Urea also contains significant carbon. Natural carbon is 98.9% 12C and 1.1% 13C. So in both cases, the odd atomic number isotopes are about 1% of the total number.

Nuclear quadrupole resonance (NOR) spectroscopy may alternatively be used in the tabletop unit sensor. NQR detects 14N without necessarily applying an external magnetic field. Other types of sensors may also be used instead of and in addition to those described.

The health status data may be stored in the memory 532 of the measurement unit 502 and then transmitted occasionally through the I/O interface 526 to a data center 504. The spectrograph is coupled to or integrated with the communications module 526. The module includes a buffer to store the measurements and a wired or wireless transmitter to send the buffered measurements through a wired or wireless interface. The communications module may store a log of the measurements locally and provide local access to a local terminal through a local interface. The measurements and/or patient identifiers may be encrypted for storage and/or transmission. This interface may be a serial bus connector, a network connector or a user interface connector. The local interface may be used to provide direct data to the patient or a clinician or any other interested party and may be used to convey alerts to the patient or any other interested local party.

The communication interface is coupled to a data center that receives and stores the measurements in a mass storage device. The mass storage may be used to store logs for multiple patients over days, months, or years. A processor unit of the data center is coupled to the logs and analyzes the data of the logs. The server may be configured to receive and store measurement in logs from more than one patient. By providing access to the data for more than one patient, patterns may be better detected and more sophisticated techniques may be used to analyze the biochemical markers.

The data center has additional processing and storage capabilities and is able to compare the results from many different patients to arrive at more accurate results. The data center may send these more refined results to a care center 506 to better care for the patient. The data center may also communicate with the patient through the measurement unit or through a computer, tablet, phone, or wearable. The measurement unit and the data center may use a cellular data modem, for example, to send SMS alerts to the patient when there is a significant change in the detected urea or when the urea reaches a high risk level.

FIG. 6 is a process flow diagram for operation of the tabletop measurement unit. After startup, the unit 502 determines at 604 whether it is time to take a measurement. This may be based on an internal clock and calendar or on instructions or commands received from an external device such as a care center 506, data center 504, physician's office, or other external agent. If it is time for a measurement then the unit alerts the patient 510 at 606. The unit may operate audible or visible signals through its user interface 526. It may also, or alternatively, send alerts to the other devices using a wireless or wired connection. The unit may send an e-mail, a text, a notification, or other indication.

The patient responds to the alert by coming to the unit and inserting an appropriate finger into the test opening 534. If the patient does not respond within some pre-determined amount of time, then the unit may provide further alerts to the patient and may also alert a care center or other external monitor that the patient is not responding. A technician or other care provider may then be sent to determine the condition of the patient. When the unit determines at 608 that the patient has inserted a finger into the measuring NMR unit then the finger is measured at 610.

In one example the unit measures the finger for 15N using NMR. Upon obtaining a clear raw result, the unit indicates at 612 to the patient that the measurement is complete. The raw measurement is analyzed by the processor of the unit at 614 to estimate a urea concentration. The urea concentration value is then stored by the unit at 616. The measurement may also be transmitted to a data center 504 for additional analysis and to back up the unit's memory. The unit may also store the raw measurement and send the raw measurement for external analysis depending on the particular implementation.

The unit further analyzes the urea concentration at 618 to determine if the result is outside of the expected safe boundaries for the urea concentration. For any normal or within-bounds result, the unit may provide a SAFE or GOOD indication to the patient. The measurement timer is restarted and the process repeats at 604. If the result is out of bounds, then the unit may provide a different type of alert at 620.

The processor unit may be configured to determine the type and detail of the alert and the parties to which the alert should be sent. The unit may notify the patient through a local terminal or the unit's user interface. The unit may also notify a clinic or hospital or other locations instead of or in addition to notifying the patient. The clinic may be coupled through a wired or wireless interface such as the Internet or a proprietary or virtual network to the data center and also to the patient's local terminal and the care center. In some cases, the data center 504 may be housed within the hospital or clinic 506. As mentioned above, the alert may cause the clinic to schedule an appointment with the patient for further analysis to determine whether the patient has a condition that requires treatment.

The out-of-bounds or other illness alert may be provided to one or more of a variety of different parties as described above. The patient may be alerted so that the patient can go to a care center to be further examined. The care center may optionally be alerted at 622 so that the patient can be scheduled for a deeper analysis or examination. The data center may use the information together with other information. After an out-of-bounds alert is generated and sent, an examination may be scheduled.

After the patient has been further diagnosed, the clinic may send the results of the diagnosis to the data center. This allows the data center to supplement the patient log. The logged measurement stored at the data center may then be associated with the diagnostic results. This allows the system to provide more accurate and personalized alerts. In addition, the measurements or a measurement pattern for one patient may be compared to patterns of other patients to improve results for the other patients. The patient identifiers in the log may be encrypted to protect the medical privacy of the patient.

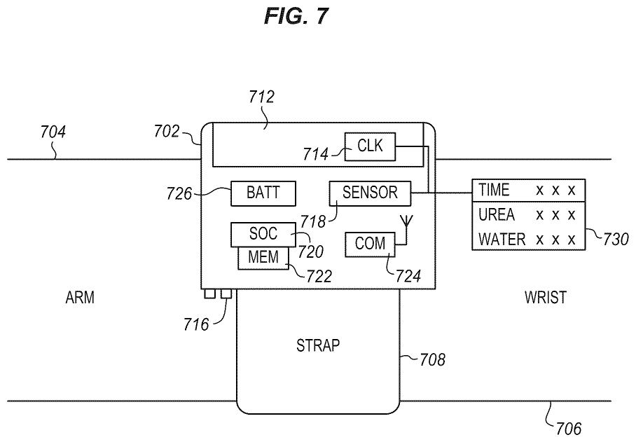

FIG. 7 is an example of an alternative measuring unit suitable for use as a wearable. The wearable measuring unit 702 allows for very frequent measurements without interfering with other patient activities. In this example, the measuring instrument may be worn on a wrist 706 or an arm 704. The measuring instrument 702 includes a power source 726, such as a battery or capacitor, a system on a chip (SOC) 720, system in a package (SiP) or other processing resource with a memory 722. A communications interface 724 may be separate or integrated into the SOC. A sensor 718 is enclosed with the other components within a case that may be attached to the wrist 706 or arm with a strap 708 as with a wristwatch. The instrument may include an electronic or mechanical watch 714. It may in addition, or alternatively include additional instruments to provide fitness strap functions and other functions. A display 712 and user controls 716 such as buttons or a touchscreen may be used to provide smartwatch functions, such as notifications, alerts, and communications as well as to allow the user to operate the measurement unit.

The sensor 718 may apply transdermal Raman spectroscopy to the wearer's wrist as a measurement method. It may measure urea with Raman spectroscopy as described above. It may also measure water concentration in order to convert the urea measurement into a concentration. Body tissue and fluids contain a significant percentage of water but this amount can vary in different parts of the body at different times. By measuring a water spectral line in addition to a urea spectral line, the ratio of those measurements can be used to provide a concentration for the urea measurement. The ratio to water may also be used to correct for coupling variations. This approach may also be used in any other embodiments described herein.

Measurements may be stored in the memory 722 and then sent externally using the communications interface 724. The measurements may also be displayed directly to the user on the display 712. The instrument may use any of a variety of wired or wireless interfaces to send the results to an external device as described below.

As a wearable device, the measuring instrument may monitor the patient's condition at any time that it is being worn. Measurements may be taken at regular intervals as determined by programming for the instrument. The instrument may be programmed to measure urea or water concentration or both at a particular interval, e.g. every 5, 30, 120, 300 minutes, etc. These measurements and timestamps 730 may be stored locally 716, stored in the memory 722, and transmitted 724. The instrument may be programmed to measure at particular times of day. The instrument may also be configured to allow the patient to command the instrument to take a measurement. The instrument may also be configured to respond to a measurement command received from an external device through the communications interface. The instrument may also use an accelerometer to identify when the patient is still and perform the measurement at that time. Possible external devices might include a smartphone, a computer, or a remote server. The instrument may be controlled using an app or other suitable interface on a smartphone or computer. The smartphone or computer may be used to provide a more extensive user interface, to provide a more complex scheduling and analysis system and to allow another person to send a message to the patient, such as SMS, chat, notification, or e-mail to request that a measurement be made.

Since the instrument is on the wrist, the monitoring may be performed at frequent intervals or even continuously, if desired. Frequent and autonomous monitoring eliminates the need to ensure that the patient remembers to take a measurement once or twice each day. Although the instrument may be configured to allow such taking of a measurement. Typically, a patient will show fluctuations in the marker levels, i.e. the urea concentration, with fluctuations in the patient's activity. Diurnal cycles, meals, exercise, and other patient activities can change the urea concentration. If the patient's activity, eating and sleeping patterns vary, then it may be difficult to obtain an accurate determination of marker concentrations through the day. Frequent or continuous monitoring allows the detection and thus correction of fluctuations in the marker levels due to diurnal cycles, meals, exercise and any other activities. As an example, the wrist-based sensor may include an accelerometer to detect activity levels including sleep and exercise and adjust measurement cycles accordingly.

The sensor in this example uses Raman spectroscopy to measure the concentration of urea and water. Raman spectroscopy allows for the detection of Mid-IR (midrange infrared) spectral features using inexpensive and high-performance near-IR or visible light sources, detectors, and optics. The laser excites the molecules in the wrist which then produce spectral lines corresponding to molecular vibrations. These may be compared to known molecule spectral lines as a way to detect and identify molecules.

As mentioned above, urea occurs in many areas of the body and the urea levels are a useful indicator for detecting illness. In a wristwatch type of device, the coupling between the sensor and the tissue or fluids being measured will vary as the position of the sensor varies. The physical coupling between the back side of the wristwatch sensor and the wrist may also vary with distance, moisture, and other factors. This may be easily compensated for using multiple measurements to normalize the results for the many variations in each measurement.

While the wrist worn sensor presents complications in size, power, and optical coupling with the wrist, it allows for convenient frequent measurements. It also allows for long measurements. Raman spectroscopy typically uses moderately high-power lasers. This allows for a stronger return signal. Common objects emit a significant amount in the Mid-IR light spectrum when at room temperature, and this background noise can reach the sensor's mid-IR detector and thereby limit the sensitivity of mid-IR measurements. A high-power laser would consume significant power from the small wrist worn power source and a cooled Mid-IR detector would consume even more power. These components would also be physically large compared to a wristwatch. The noise issues may also be compensated by taking longer measurements.

A lower power laser reduces the amplitude of the return optical signal compared to the background noise. This causes a signal-to-noise performance penalty. A longer measurement time allows for more background noise to be collected by the optical return signal detector. The detector background noise is proportional to time. This causes a further signal-to-noise performance penalty.

On the other hand, the return optical signal is also proportional to the laser power multiplied by the time. In order to compensate for the increased noise, the fluctuations in noise may be analyzed. While the signal and noise both increase in direct relation to the time, the fluctuation in the noise increases only proportional to the square root of the time (sqrt (time)).

This square root relationship allows the laser power to be reduced for eye safety, power conservation, and size reduction while still obtaining useful measurements. As an additional safety feature, an interlock may be provided that determines when the sensor is near a wrist. When the sensor is removed or is too distant from the wrist, then the laser is turned off. The proximity sensor may be the optical sensor of the Raman spectrometer or a separate proximity sensor may be used on the device. The proximity sensor may be mounted on the back of the case, for example facing the user's wrist.

In use, the controller of the measurement unit receives a measurement command from a software timer, a user command, or an external device. The controller drives the sensor to determine whether the instrument is next to a wrist. If so, then it drives the sensor to generate an excitation signal and measure the emitted light from the patient's wrist. The measurement is analyzed by the processor and then it is stored in memory. The instrument then finishes the process and returns to a start of the process. In a separate process, the instrument transfers the stored data to external devices such as a smartphone, computer, or server. This process may be conducted using conventional protocols and the transferred data may then be used to determine illness, schedule care, or in other ways.

As an alternative to the wrist watch form factor, the wearable measuring unit may be in the form of other conventional garments or accessories. As an example, the unit may be supported by a belt around the patient's waist. The sensor may be connected by an optical fiber to a small sensor head which is attached to the body by the belt. Alternatively, the sensor may be attached to the abdomen, back, leg, arm, or wrist, by either a band of some type, for example an elastic band, or in another way such as by adhesive tape.

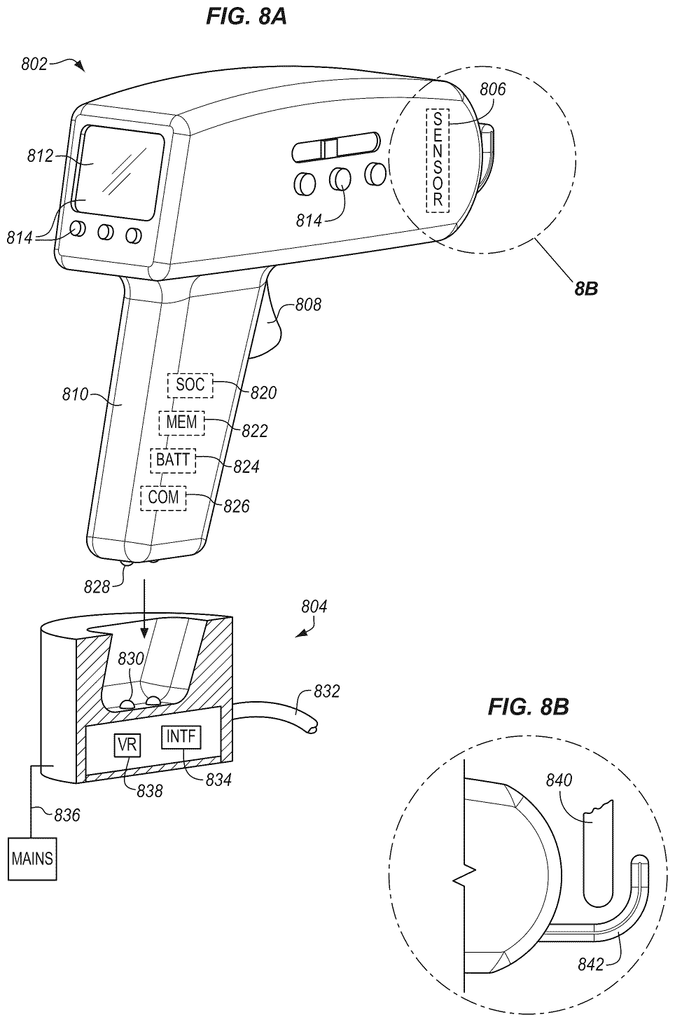

FIG. 8A is a diagram of an alternative measuring unit suitable for use as a portable handheld instrument. The portable handheld instrument allows easier use by a technician and allows for other types of measurements to be made. In this variation, the portable handheld unit 802 includes a power source 824, such as a battery or capacitor, a processor 820, such as a SOC, SiP or discrete controller, a memory 822 which may or may not be a part of the SOC, a communications interface 826 and a sensor 806. These are all enclosed within a case that can easily be held in the hand using a handle 810 of the case so that a Raman spectroscopy instrument 806 may be directed to a patient for measurement of a suitable health marker such as urea concentration. The instrument may also include additional instruments to provide blood oxygen, temperature, and other measurements. A display 812 and user controls 814 such as buttons or a touchscreen may be used to provide additional control and communication functions, such as notifications, alerts, and communications.

The sensor may apply transdermal Raman spectroscopy to a patient's earlobe, forehead or other suitable location as a measurement method. FIG. 8B is an enlarged side view of the sensor part of the handheld instrument suitable for an earlobe 840 for which a clip 842 may be provided to allow an operator to hold the sensor fixed against the earlobe. This allows the laser to be positioned on one side of the earlobe and the near-IR (NIR) sensor to be positioned on the opposite side of the earlobe for example in the clip 842 so that a transmission measurement may be used. The transmission measurement allows the pump attenuation to be measured and then used to normalize the urea measurement. As an alternative, the measurement instrument may be configured for a contact measurement with the laser and sensor on the same side. This is also suitable for forehead, wrist and other measurement locations.

In some examples, the sensor measures urea with Raman spectroscopy as described above. It may also measure water concentration in order to convert the urea measurement into a concentration. Body tissue and fluids contain a significant percentage of water but this amount can vary in different parts of the body at different times. By measuring a water spectral line in addition to a urea spectral line, the ratio of those measurements can be used to provide a concentration for the urea measurement. The ratio to water may also be used to correct for coupling variations. In other examples, a different sensor may be used.

The measurements may be stored for later transfer to an external device. In this example, the instrument includes a base of the handle 810 with a docking connector 828 that attaches to a dock 804. The dock may include mating connectors 830 to receive the base of the handheld unit. The dock may include a USB, Ethernet, or other suitable data connector 832 to provide power to recharge the instrument and to transfer data between the instrument and a connected terminal (not shown). A separate power supply or voltage regulator 838 may alternatively be used to provide mains power to the handheld unit. A data interface 834 between the connectors and the cable may be used to couple the handheld unit to the connected terminal through the dock. Alternatively, wireless interfaces may be used for data transfer.

Using the dock, the measurements may then be transferred to the connected terminal and any updates may be transferred to the instrument. As examples, the dock may be used to transfer software updates, patient information and spectroscopy calibration data to the instrument. While a dock is shown, similar functionality may be accomplished using a simple USB connector or other type of power and data connector. The dock connection may be electrical, inductive or otherwise.

This handheld unit easily accommodates a significant battery, a powerful laser and a cooling system for the return optical sensor of the Raman spectroscope. The sensor may be carried by a technician at a hospital, clinic, or other care facility to perform measurements on many different patients before being recharged. As an example, a technician may use the instrument in a nursing home so that the technician visits each patient each day to monitor their health. As another example, a technician may visit post-surgical patients in a hospital, at a post-surgical recovery facility, or at home each day to monitor their recovery for complications

At the end of the day or at the end of performing rounds, the instrument may be attached to the dock to download all of the measurements into a computer through a USB connector. Any service or software updates may also be uploaded to the instrument and the instrument's battery may be recharged. Alternatively, any other suitable wired or wireless connection may be used.

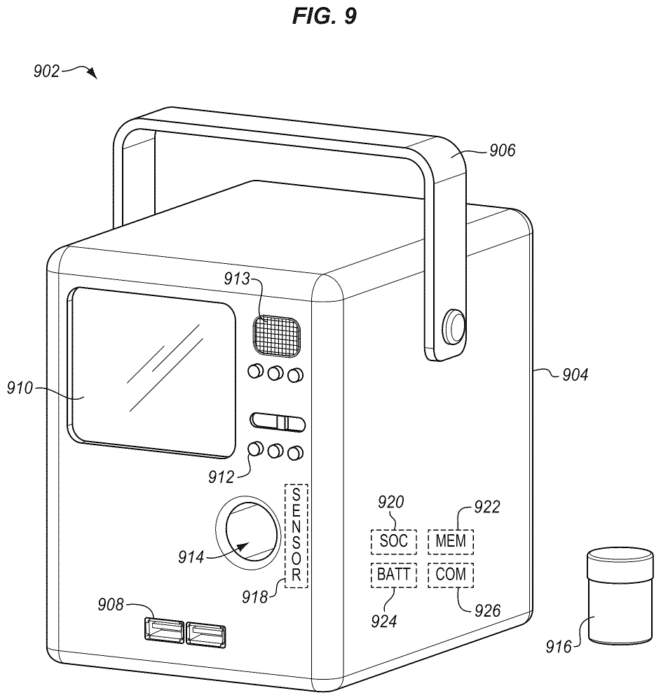

FIG. 9 is a diagram of an alternative measuring unit suitable for use as a portable tabletop instrument for use with collected samples. The tabletop instrument allows samples to be collected and measured by bringing patients to a fixed location or by bringing the instrument to patients. Like the other examples, it is suitable for use at home, at a clinic, at a hospital, or in any other setting. The proper collection of samples may be more easily performed by a technician, but a patient may prefer to perform the measurements at home.

The tabletop instrument 902 may be a fixed or a portable device. In this example it includes a housing 904 with a handle 906 for carrying the instrument to different locations. A user interface includes a display 910 and buttons or switches 912. A touchscreen or any other suitable interface may also be used. A speaker 913 may be used for audible alerts or other notifications. The housing also includes a tube or cylindrical sleeve 914 for receiving samples for analysis and a port 908, such as a USB port for power and data transfer.

The functional components inside the housing 904 may be similar to those of the other examples and include an SOC 920, memory 922, battery 924, and communications interface 926.

A sensor 918, such as a Raman spectroscopy sensor may be used to analyze the urea concentration samples placed in the sample tube 914. A sample container 916 may be used to hold saliva samples or any other type of samples. The samples may be collected and analyzed in disposable or reusable sample containers 916 and placed in the sensor tube for analysis. The sample containers may be pre-loaded with a wetting agent to reduce bubbles which may interfere with the measurement. Bubbles may be an issue for optical measurements because bubbles strongly scatter light. The wetting agents may be used to lower the surface tension of water in the saliva and allow bubbles to float to the surface. The sensor may provide the data to the SOC to analyze the data and indicate any alerts on a built-in display.

The interior components and functions may be similar to the handheld unit or the tabletop finger sensor, but this device may be configured to fit within a larger and heavier form factor. The larger form factor may allow for a more powerful processor, longer lasting battery, and a more complete suite of communication interfaces, such as data and voice interfaces.

The tabletop unit may be used in nursing homes or during home visits and the larger form factor with greater power, communications and battery life is particularly suitable for traveling to more remote locations. The tabletop unit may be adapted to measure a finger inserted into the tube. It may also be adapted for use with urine. Bodily fluids such as saliva and urine provide a stronger signal that is easier to measure than the transdermal measurement described above. Compared to other body fluids, saliva is easy to produce and easy to handle. Like other body fluids, saliva urea levels track with body urea levels.

In use, a technician or the patient presents a sample container which is then filled with saliva. The container is inserted into the measurement tube and the measuring instrument is activated. The user interface may be used to enter information about the patient or any other suitable data. The instrument then measures the sample for urea concentration or some other suitable marker and analyzes the result. The results may alternatively be sent to an external device for analysis through the USB interface or through a wireless interface. The analysis, such as an alert may then be displayed on the screen. For a negative result, the patient may schedule an examination through a separate telephone or computer terminal or, in some configurations, directly with the measurement instrument.

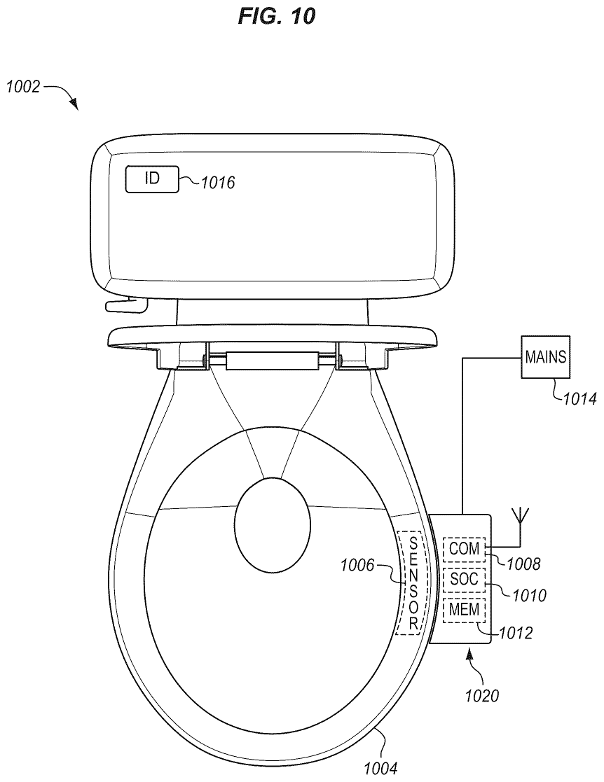

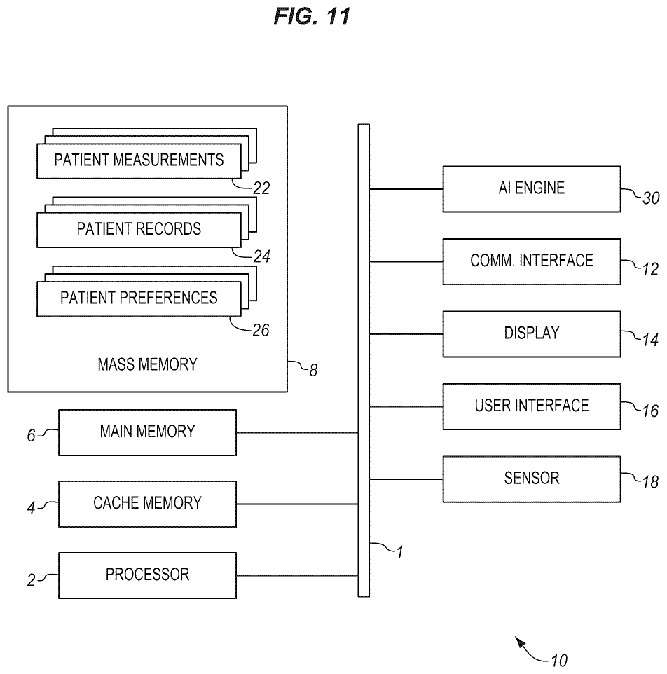

FIG. 10 is a diagram of an alternative fixed liquid sample collection device suitable for as a measuring unit for detecting urea. The fixed sample collection provides ease for patients to use provided that other functions are sufficiently automated. The instrument 1020 is integrated with a toilet 1002 and attached to or built into the toiled bowl 1004. Infrared spectroscopy or any other suitable technique may be used to analyze urine before the toilet is flushed. To enhance accuracy, the volume of urine may be determined for use in concentration calculations. A Wi-Fi or wired connection may be used to report the measurements to a remote server for analysis. Alternatively, the analysis capabilities described above may be incorporated into the measurement instrument 1020 in a similar way.

The measurement instrument 1020 includes a power source connection 1014, such as a connection to the mains or a battery or capacitor may be used. The instrument further includes a processor 1010, such as a SOC, SiP or discrete controller, a memory 1012 which may or may not be a part of the SOC, a communications interface 1008 and a sensor 1006. These may be enclosed within a case or integrated into the components of the toilet.

The instrument may be configured to determine when a patient has deposited urine and then activate the sensor before the bowl is flushed. The same urine detection may also be used to determine how much urine was added to the bowl. This may be used to determine the relative amount of urine to water in the bowl. If urea concentration is being determined, then it is useful to compare the added urine from the existing amount of water. Suitable liquid level sensing technologies include pressure sensors, capacitive sensors, optical sensors, and ultrasonic distance measurements to determine a position of the top surface of the liquid in the bowl. These sensors may be integrated into the bowl or attached to the bowl as an accessory.

Because the sensor is installed in a fixed location, mains power may be used. As a result, more accurate and effective higher power consumption components may be used. The sensor may use a mid-IR light source and a cooled detector for mid-IR spectroscopy. This would require more power than some of the variations described above. The mid-IR light source may be a suitable laser and the detector may be a silicon photodetector sensor with appropriate light filter.

In use, the ‘smart toilet’ would likely be shared by different people. If the ‘smart toilet’ is installed in a clinic, a hospital, or a nursing home, then the cost and maintenance of the measurement unit may be shared across many different users. In some such applications, there will be multiple samples collected in a day so that some of the advantage of the wearable measurement instrument may be realized. To distinguish different users, the user may enter an access code or provide some other method of identification. This may include RFID codes from bracelets or garments, a radio interface on a personal door key, a smartphone authentication signal, or some type of autonomous identification, such as facial recognition. In FIG. 10 an ID unit 1016 may be an RFID tag reader, camera, or other signal receiver to identify a person.

FIG. 11 is a block diagram of a computer system 10 representing an example of a system upon which features of the described embodiments may be implemented, such as the computing systems of FIG. 1, the monitor, measuring instruments, local terminal, server, data center, or clinic in their various illustrated embodiments. These systems may include or be implemented as such a computer system, depending on the implementation and associated equipment. The computer system includes a bus or other communication means 1 for communicating information, and a processing means such as one or more microprocessors 2 coupled with the bus for processing information. The computer system further includes a cache memory 4, such as a random access memory (RAM) or other dynamic data storage device, coupled to the bus for storing information and instructions to be executed by the processor. The main memory also may be used for storing temporary variables or other intermediate information during execution of instructions by the processor. The computer system may also include a main nonvolatile memory 6, such as a read only memory (ROM) or other static data storage device coupled to the bus for storing static information and instructions for the processor.

A mass memory 8 such as a solid state disk, magnetic disk, disk array, or optical disc and its corresponding drive may also be coupled to the bus of the computer system for storing information and instructions. The computer system can also be coupled via the bus to a display device or monitor 4 for displaying information to a user. For example, graphical and textual indications of installation status, operations status and other information may be presented to the user on the display device. A user input device 16, such as a keyboard with alphanumeric, function and other keys, a cursor control input device, such as a mouse, a trackball, trackpad, or cursor direction keys, buttons, sliders, wheels, and a touchscreen, etc. can be coupled to the bus for communicating direction information and command selections from a user to the processor. In some implementations, one or more sensors 18 for measuring catabolic or other markers is attached to the bus 1 and may operate autonomously or under the control of the processor.

A communications interface 12 is also coupled to the bus. The communication device may include a wired or wireless modem, a network interface card, or other well-known interface devices, such as those used for coupling to Ethernet, token ring, or other types of physical attachment for purposes of providing a communication link to support a local or wide area network (LAN or WAN), for example. In this manner, the computer system may also be coupled to a number of clients or servers via one or more conventional network infrastructures, including an Intranet or the Internet, for example. The communications interface may additionally or alternatively incorporate wireless links as described above.

The mass memory 8 may be used to store data of several patients as discussed above. The data may take the form of tables or any other structure. In this example, a patient measurement table 22 contains measured values for one or more patients collected over time or shared from an external source. There may be different tables for different types of measurements or markers, such as urea, lactic acid, proteins, alanine cycle markers, etc., or for different types of monitors, such as finger, forehead, wrist, bodily fluid, etc. There may also be tables for other measures, such as movement, pulse rate, blood oxygen, etc. A patient records table 24 contains other medical or personal data about the table that may be required by a clinic, server, doctor or other participant in the system. Again, there may be different tables for different patients. A patient preferences table 26 contains various operational or care preferences depending upon the use of the system. This may include display configuration, times for monitoring, contact preferences, preferred appointment times or any other suitable preference.

The described tables may be stored as two-dimensional tables, as text files with metadata, or in any other desired way. The data from the patient measurement table is collected and analyzed by the processor in response to commands from the user interface 16 as indicated by the preferences tables 26. The system may also be operated or accessed remotely through the communications interface 12.

The system of FIG. 11 optionally further includes an AI (Artificial Intelligence) engine 30. This may be implemented in dedicated hardware using parallel processing or in the processor 2 or using some combination of resources. The AI engine may also be external to a server system 10 and connected through a network node or some other means. The AI engine may be configured to use historical data accumulated by the server system to build a model that includes weights and criteria to apply to the analysis processes. The model may be repeatedly rebuilt using the accumulated data to refine and increase accuracy. Other types of analysis systems may be used alternatively or in addition to those shown.

The computer system is shown as discrete components attached to a bus, however, one or more of the components may be combined and others added. As an example, some or all of the components may be combined into one or more SiPs, or SoCs or some combination of these. While many of the same basic types of components are used, an autonomous wrist monitor, a rechargeable handheld monitor and a server center may be constructed using very different hardware implementations.

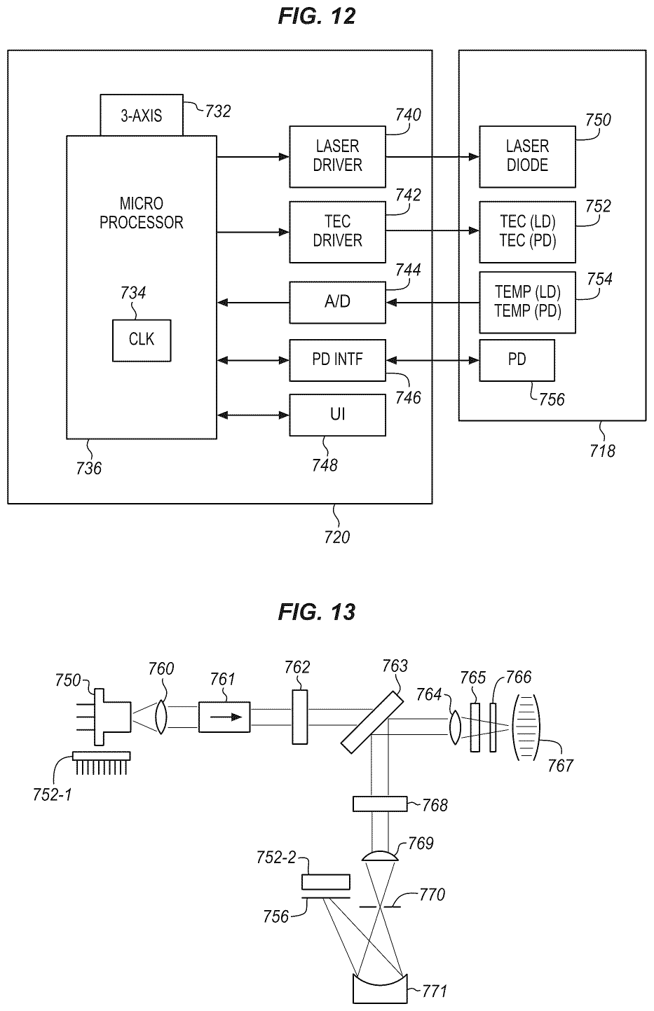

FIG. 12 is a diagram of components of the SOC 720 and sensor 718 according to some embodiments. Among other components, the SOC optionally includes a motion sensor 732 such as a 3-axis accelerometer and a real time clock 734. This may be used to determine whether the patient is in an active or relaxed state and to assess suitable conditions and times for activating the sensor for measurements. The microprocessor is coupled to a power supply 726, communications modem or modems 724 such as a Bluetooth, GSM/GPRS, Wi-Fi, or LTE modem and other components as mentioned above. As mentioned above the basic configuration of FIGS. 7 and 8 may be adapted to suit other form factors for wearable and independent devices.

The microprocessor is able to drive other components within the SOC or optionally external to the SOC to operate the sensor. A laser driver 740 generates power under control of the microprocessor to cause the laser diode (LD) 750 of the sensor to generate suitable light for making a measurement and for calibration. A thermoelectric cooler (TEC) driver 742 generates power to drive one or more TECs 752 on the sensor. The coolers may be associated with the LD 750, the photodiode (PD) light sensor 756, and other components of the sensor. The TECs may be controlled independently of each other to allow precise control of the sensor components. A thermal sensor interface 744 receives readings from temperature sensors 754 of the sensor and provides these to the microprocessor. The microprocessor may be configured to use this data to control the coolers, the LD and the PD. A photodiode interface 746 allows the timing, scan rate, and other actions of the PD 756 to be controlled. It also provides the PD data to the microprocessor for analysis and to be logged. The microprocessor also has a user interface module 748 for connection to the display 712 and user controls 716.

The system of FIGS. 7 and 12 may be operated in any of a variety of different ways to suit particular types of sensors, biochemical markers, and patient tissues. More or fewer components than those shown may be used to implement the operations. In one example, the system may be configured to use the inertial sensor 732, such as the three-axis accelerometer, to identify when the patient is still. The microprocessor's real time clock may then be used to identify when it is time to acquire a measurement. Measurements may be made based on a timer, a time of day or another schedule.

Next, the photodetector thermoelectric coolers (TECs) 752 are activated and stage 1 and stage 2 temperature sensors 754 are read to regulate the cooler drive currents. The coolers and sensors are used together to maintain the photodetector 756 at an optimum or pre-determined operating temperature. A variety of different control techniques may be used. In one example, a proportional-integral-differential controller technique is applied. At about the same time, the laser diode thermoelectric cooler 752 is activated and the laser diode temperature sensor is read to regulate the laser diode temperature to an optimum or pre-determined temperature using the same or similar control methodology.

The laser diode 750 of the sensor is then activated by the microprocessor. The microprocessor may have laser diode temperature and drive current setpoints to ensure accurate operations. These are initiated to initial or pre-determined setpoints. The laser and coolers are operated until the initial values are achieved and stabilized. The PD interface 746 operates the PD 756 to acquire an initial spectrum from the tissue and this data is saved in the memory 722 or in a temporary cache.

Optionally to achieve higher accuracy, the laser diode temperature and drive current can be changed to a second temperature setpoint and drive current setpoint to shift the laser light frequency. The microprocessor then waits for current and temperature to stabilize with the second setpoints. The PD interface then causes the PD to acquire spectra using the photodetector and to save this additional data.

After two acquisitions, the spectra data may be analyzed to determine if the data quality meets a threshold or standard expectation. The process of setting temperature and drive current and acquiring spectra is repeated until a full measurement cycle has been completed. The microprocessor then deactivates the laser, the laser thermoelectric cooler, and the photodetector thermoelectric coolers. The obtained data may then be analyzed. While coolers are described, such as Peltier coolers, simpler heaters or other thermal systems may be used. Also the output light frequency of the laser may be adjusted by changing other operational parameters of the laser instead of or in addition to the temperature.

In examples, the initial value sweeps are averaged together; shifted sweeps are averaged together a more accurate value can then be obtained by subtracting the initial value average from the shifted average. By using two different LD light frequencies and by taking multiple scans of the tissue, many sources of error and interference can be eliminated. Additional sweeps may be taken at additional frequencies. Other simpler or more complex techniques may be used to improve signal quality.

In this Raman spectrometer, the Raman spectral line strength may be determined based on a final sweep value. In one example, a partial least squares analysis is used to arrive at the line strength. The results may then be logged and communicated to external components including a touch screen, as described above. In addition or instead, an integrated GSM/GPRS modem may be used to upload measurement results to a cloud server. The measurement may then be reset for the next cycle and this process may be repeated when the patient is sufficiently still. The measurement interval may also be adjusted based on a risk algorithm and the measurement results.

FIG. 13 is a diagram of the optical system of the Raman sensor of FIG. 12 in more detail. More or fewer optical elements may be used than shown in this diagram. The laser diode 750 is thermally coupled to an LD thermoelectric cooler 752-1, such as a Peltier cooler. The cooler stabilizes and tunes the LD by controlling its temperature. Alternatively a simpler resistive heater may be used to heat but not cool the LD. Other devices may be used to modify other laser parameters instead of or in addition to temperature. The laser may be a laser diode of a suitable frequency for Raman spectroscopy of the appropriate type of tissue. Suitable infrared, red, or green LDs may be used among other types of compact LDs. For tabletop units gas and other types of lasers may be used instead.

The laser illumination is coupled into a collimating lens 760 and optionally passed through an optical isolator 761. The isolator attenuates reflected LD light that is returning to the laser from the tissue or other optical elements. If reflected light reaches the LD, then it may change the energy of the LD changing the amplitude or the frequency of the LD output. Optionally a second filter, such as an amplified spontaneous emissions (ASE) filter 762 blocks or absorbs other light emitted from the LD that would otherwise add noise to the Raman signal.

A dichroic beam splitter 763 passes the Raman pump signal from the LD to the tissue 767. Energy from the tissue is reflected in the direction of the photodetector 756. After the filters 761, 762, and beam splitter 763, the collimated 760 LD 750 illumination is directed and focused by another lens or lens system 764 into the patient tissue 767. This lens focuses the pump signal down to a small tissue area to increase the Raman scattering within the small area.

The focused beam passes through a window 765 of the sensor that protects the internal components of the sensor from dust, moisture and other contaminants. The window may be configured to provide an hermetic seal against ambient moisture to reduce the dew point of the optical system. It may be optically powered. A spacer 766 is provided between the window 765 and the tissue 767 to protect the window from the tissue. The spacer also controls the distance between the focusing lens 764 and the tissue. This distance determines the position of the focus point of the pump signal within the tissue. In the example of FIG. 7, the tissue is an arm or wrist. However the tissue may be any other tissue or a sample that is extracted such as urine, sweat, or saliva as discussed above.

According to Raman spectrometry principals, the tissue that is illuminated by the pump signal absorbs the pump signal energy and emits photons at different frequencies or wavelengths that are determined by the condition and composition of the tissue. This emitted light is in part emitted back in the direction of the pump signal across the spacer 766, through the window 765 and collimated by the focusing lens 764 to the beam splitter. The different wavelength of the light emitted from the tissue causes the light to be reflected and not transmitted by the beam splitter toward the PD 756.

The emitted light passes through an optional filter 768 to block or absorb any additional pump signal light in the optical path. This is followed by an optical system to direct the emitted light to the PD 756 which also has thermal control system, such as a Peltier thermoelectric cooler 752-2 or a simpler heater. In this example the optical system is configured to be compact and to direct collimated light across the surface of the PD with minimal attenuation. This system has a focusing lens 769 optically coupled to the reflection from the beam splitter, an optical slit 770, and a curved diffraction grating (DOE) to reflect the emitted light off the optical axis of the beam splitter toward the PD. A variety of other optical systems may be used instead for other physical configurations.

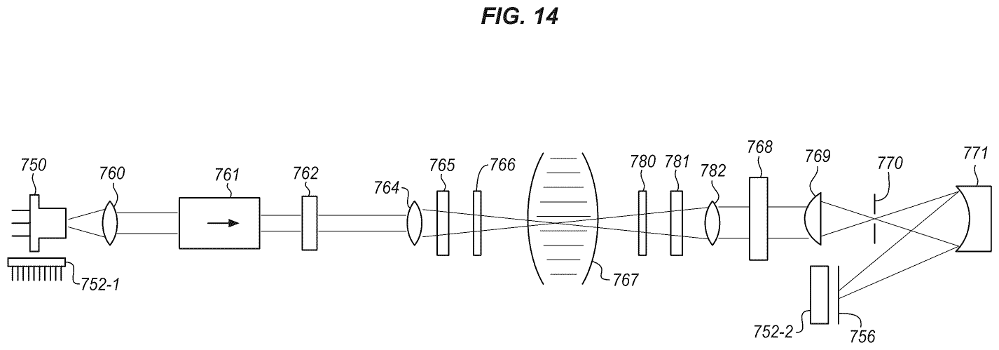

FIG. 14 is a diagram of an alternative optical system for a Raman sensor such as that shown in FIG. 8B in which the PD is on the opposite side the tissue from the LD. This system has the same optical elements as in FIG. 13 except that the beam splitter is removed. Instead, the emitted light from the tissue is received from another direction. A second spacer 780 positions a window 781 to transmit the emitted light from the tissue to a lens 782 that collimates the emitted light to a pump signal filter 768 as in the FIG. 13 example. The emitted light is then transmitted to the PD 756 as in FIG. 13.

Throughout the specification, reference is made to various processors, controllers, SOCs, SiPs and other computational components. The appropriate components may be selected based on the power demands, the processing demands, and cost constraints. Accordingly, any one of the controllers, processors etc., may be an FPGA (Field Programmable Gate Array), an ASIC (Application Specific Integrated Circuit) designed for the particular purpose, a microcontroller, it may be a simple embedded processor with appropriate programming, a complete microprocessor with internal program memory and multiple processing cores, or any other suitable type of processor. The controller or processor may include or be packaged with memory, communications, display controller, graphics, user input and other components. The illustration of each of these components is not intended to require any particular hardware configuration but is to show functionality that is particularly of interest to the described embodiment.

The embodiments described herein include communications interfaces. In some cases, the measurement instrument may be used to simply provide information on a display. A human may then notify an appropriate person of a measurement result or an analysis performed directly by the instrument. In other cases, results are sent to data centers, clinics or various individuals. Any of a variety of different interfaces may be used. Wired interfaces may include USB, Ethernet, or another suitable wired interface. Wireless interfaces may include Bluetooth, ZigBee, Wi-Fi, cellular, such as LTE, GSM, GPRS or any of a variety of other wireless interfaces to send data to external components.

For some embodiments power conservation is important in order to conserve battery power. For the handheld or wrist-based instrument, the data may be stored and then transmitted to a wired interface. This has the advantage of lower power consumption but it delays the sending of the data. In other examples, the data may be transmitted using a suitable short-range low power system, such as Bluetooth, to another device such as a smartphone or computer that then forwards the data to remote external data centers or clinics. The smartphone or computer acts as a repeater in this instance. With recent developments for IoT (Internet of Things) additional low power transmission protocols are being developed for Wi-Fi HaLow and 5G LTE and any of these may alternatively be used as low-cost components become available.

As mentioned above, in some instances, the smartphone or computer acts as a repeater between the measurement instrument and remote nodes. However, the smartphone or computer may also act as a data processor and analyze the data to determine any suitable alerts. The smartphone or computer may be used to compile results over time and receive suitable data so that an accurate analysis may be provided locally to the user. The smartphone or computer may also be used as part of the user interface. A smartphone or computer app may allow for more detailed measurement information or more detailed control over the measurement instrument. A smartphone or tablet may be used as a portable supplemental control interface for operating the measurement instrument.

A lesser or more equipped sensor, monitor, terminal, clinic, or server system than the examples described above may be used for certain implementations. Therefore, the configuration of the system will vary from implementation to implementation depending upon numerous factors, such as price constraints, performance requirements, technological improvements, and/or other circumstances.

Many of the operations described herein may be performed under the control of a programmed processor, such as central processing unit, a microcontroller or by any programmable or hardcoded logic, such as Field Programmable Gate Arrays (FPGAs), TTL logic, or Application Specific Integrated Circuits (ASICs), for example. Additionally, the methods of the present invention may be performed by any combination of programmed general purpose computer components and/or custom hardware components. Therefore, nothing disclosed herein should be construed as limiting the present invention to a specific combination of hardware components.

The present description presents the examples using particular terms, such as monitor, marker, clinic, patient, doctor, health, illness, sign, symptom, etc. These terms are used to provide consistent, clear examples, however, the present invention is not limited to any particular terminology. Similar ideas, principles, methods, apparatus, and systems can be developed using different terminology in whole, or in part. In addition, the present invention can be applied to ideas, principles, methods, apparatus, and systems that are developed around different usage models and hardware configurations.

In the present description, for the purposes of explanation, numerous specific details are set forth in order to provide a thorough understanding of the present invention. However, the present invention can be practiced without some of these specific details. In other instances, well-known structures and devices are shown in block diagram form. The specific detail can be supplied by one of average skill in the art as appropriate for any particular implementation.

Embodiments of the present invention include various steps, which can be performed by hardware components or can be embodied in machine-executable instructions, such as software or firmware instructions. The machine-executable instructions can be used to cause a general-purpose or special-purpose processor programmed with the instructions to perform the steps. Alternatively, the steps can be performed by a combination of hardware and software.

Embodiments and portions of the present invention can be provided as a computer program product that can include a machine-readable medium having stored instructions thereon, which can be used to program a computer (or other machine) to perform a process according to the present invention. The machine-readable medium can include, but is not limited to, floppy diskettes, optical disks, CD-ROMs, and magneto-optical disks, ROMs, RAMs, EPROMS, EEPROMs, magnet or optical cards, flash memory, or any other type of medium suitable for storing electronic instructions.

Although this disclosure describes illustrative embodiments of the invention in detail, it is to be understood that the invention is not limited to the precise embodiments described. The specification and drawings are, accordingly, to be regarded in an illustrative rather than a restrictive sense. Various adaptations, modifications and alterations may be practiced within the scope of the invention defined by the appended claims.

1. A portable catabolic marker measuring unit comprising:
a case;
a catabolic marker sensor in the case to measure a concentration of a catabolic marker in a user in vivo; and
a proximity sensor in the case to determine whether the catabolic marker sensor is near the user, wherein the catabolic marker sensor measures the concentration in response to the determining that the catabolic marker sensor is near the user.

2. The unit of claim 1, further comprising an accelerometer in the case to identify when the patient is still, wherein the catabolic marker sensor measures the catabolic marker in response to the identifying that the user is still.

3. The unit of claim 2, wherein the proximity sensor is the catabolic marker sensor.

4. The unit of claim 1, further comprising a handle to direct the catabolic marker sensor to the user.

5. The unit of claim 1, further comprising a strap attached to the case for moving the unit.

6. The unit of claim 5, wherein the strap is configured to attach the case to a wrist of the user.

7. The unit of claim 1, further comprising a user interface attached to the case, the user interface configured to display a time of day.

8. The unit of claim 1, further comprising a user interface attached to the case, the user interface configured to provide alerts and to allow the user to cause the catabolic marker sensor to measure the catabolic marker.

9. The unit of claim 8, wherein the user interface is on a surface opposite the catabolic marker sensor.

10. The unit of claim 1, wherein the catabolic marker sensor is configured to measure a water concentration in addition to the concentration of the catabolic marker and wherein the unit converts a measure of the catabolic marker to a catabolic marker concentration using the water concentration.

11. The unit of claim 1, further comprising a memory in the case to store the concentration with a corresponding timestamp.

12. The unit of claim 11, further comprising a wireless communications interface configured to send concentrations from the memory to an external device.

13. The unit of claim 1, further comprising a processor in the case having programming to cause the catabolic marker sensor to measure the concentration at regular intervals of time.

14. The unit of claim 13, wherein the regular intervals correspond to particular times of day.

15. The unit of claim 1, wherein the processor is configured to cause a user interface of the unit to generate an alert for the catabolic marker concentration.

16. The unit of claim 1, wherein the catabolic marker sensor comprises a midrange infrared laser configured to excite molecules in the user to produce spectral lines and a photodetector to detect the spectral lines for comparison to known molecule spectral lines.

17. A method of a portable catabolic marker measuring unit comprising:
determining whether a catabolic marker sensor in a case of the unit is near a user using a proximity sensor in the case of the unit; and
measuring a concentration of a catabolic marker in the user in vivo by the catabolic marker sensor in response to the determining that the catabolic marker sensor is near the user.

18. The method of claim 17, further comprising:
measuring the concentration at regular intervals corresponding to particular times of day;
analyzing the results to determine whether an alert condition is presented; and
presenting an alert on the case in response to determining the alert condition.

19. A portable catabolic marker measuring unit comprising:
a case;
means for determining whether a catabolic marker sensor in the case of the unit is near a user using a proximity sensor in the case of the unit; and
means for measuring a concentration of a catabolic marker in the user in vivo by the catabolic marker sensor in response to the determining that the catabolic marker sensor is near the user.

20. The method of claim 19, wherein the means for measuring the concentration measures the concentration at regular intervals corresponding to particular times of day, the method further comprising:
means for storing the concentration corresponding to particular times of day;
means for analyzing the stored concentrations to determine whether an alert condition is presented; and
means for presenting an alert on the case in response to determining the alert condition.
